(19) United States
(12) Patent Application Publication
(10) Pub. No.: US 2003/0091625 A1
(43) Pub. Date: May 15, 2003

(54) Method and device for producing compression coated tablets

(76) Inventors: Madhusudan Hariharan, Evanston, IL (US); Vishal K. Gupta, Northbrook, IL (US); Robert M. Noack, Grand Rapids, MI (US); Torkel Gren, Uppsala (SE)

Correspondence Address: PHARMACIA CORPORATION, GLOBAL PATENT DEPARTMENT, POST OFFICE BOX 1027, ST. LOUIS, MO, 63006, US

(21) Appl. No.: 10/179,588

(22) Filed: Jun. 25, 2002

Related U.S. Application Data
(60) Provisional application No. 60/300,629, filed on Jun. 25, 2001.

(51) Int. Cl. A61K 9/20; B27N 3/00

(52) U.S. Cl. 424/465; 264/109

(57) ABSTRACT

A method is disclosed for producing coated compressed tablets, using at least one upper punch. The upper punch can be a single punch with a hollow shaft containing a movable rod with a tip that is extended or retracted as the rod moves within the shaft. Alternatively, two solid upper punches are used, one with an extended tip and the other with a retracted tip. An upper punch with an extended tip is designed to produce cup-shaped compacts of excipient and/or polymeric tablet coating material, when inserted into the die. Core material is inserted into each cup. Additional coating material is then placed on top of the cup and core, within the die, and compressed with an upper punch with a retracted tip. The method and device of the present invention enables one to produce tablets from poorly compactable substances.

This application claims the benefit of U.S. provisional patent application serial No. 60/300,629, filed Jun. 25, 2001.

### FIELD OF THE INVENTION

The present invention relates to compaction of particles into coherent bodies, such as tablets. In particular, the present invention relates to methods for compression coating. The field of the present invention also includes devices for compression coating and compression coated compositions, such as coated tablets or partially coated tablets.

### BACKGROUND OF THE INVENTION

A new bioactive substance is often first formulated as a free-flowing granulation for encapsulation within hard gelatin capsules. Over the course of clinical development, the drug-containing granulation is often modified to make it compactable into a tablet product. For a summary of many known methods of manufacturing tablets, see Remington's Pharmaceutical Sciences, 16ₜₕ edition, Arthur Osol et al., eds. (Mack Pub. Co., 1980), pp. 1553-1576, incorporated by reference herein. The tablet product may be subsequently coated for taste-masking, identification or other purposes. For a summary of many known methods of tablet coating, see Remington's, supra, at pp. 1585-1593, incorporated by reference herein.

Modem compression-coated products are made on specialized equipment in two separate operations: manufacture of core tablets on a traditional tablet press; and the subsequent application of a compression-coating granulation. Such compression-coating production processes require relatively complex machinery to encapsulate the core tablet within an outer compression-coating material.

A Manesty Dry Cota tablet press uses a slightly different method of making compression-coated tablets in which the core and coated tablet are made simultaneously, but, in separate sections of the press before being combined and compressed. See Gunsel, W. C. “Compression-Coated and Layer Tablets”, in Pharmaceutical Dosage Forms: Tablets, Volume 1. H. A. Lieberman and L Lachman (eds.), Marcel Dekker, New York, pp. 187-224 (1980). The machinery is a relatively complex double turret press (basically two conjoined tablet presses) in which the core is formed on one side of the press and then transferred to the other side where the compression coating is applied.

More recently, modifications of the above-cited methods and materials for producing compressed coated tablets have been developed. WO 01/15889 A1, for an invention by Gunter Voss, discloses use of a tablet press with a compression chamber and an upper and a lower punch. FIG. 1 of that publication shows an apparatus of the invention, that includes an upper stamp, a lower stamp, and a compression chamber, wherein a tip end of the upper stamp and a top end of the lower stamp are designed to fit into an opening that extends from one end to the other of the compression chamber. The top end of the lower stamp includes a projection that is “closed in itself” (Abstract). In the embodiment of that invention illustrated in FIG. 1, the tip of the upper stamp includes a rod-like structure emerging from the tip surface directed toward the compression chamber. Compressed tablets are made using the apparatus disclosed therein by either coating the surface of the lower punch surrounded by the projection in the lower punch with core material and then adding coating material thereto prior to compression with the upper and lower stamps; or tablets are made by adding coating material to the lower punch in the compression chamber, and adding core material to the surface of the coating material surrounded by the projection of the lower punch, prior to compression.

Another method of producing coated tablets is disclosed in U.S. Pat. No. 2,001,0123, for another invention by Gunter Voss. That method uses a tablet press that has at least one compression chamber with an upper and a lower punch and a feeding device for tablet cores. Coating for the lower half of the tablet is placed in the chamber, precompressed, core material in the form of a “pasty preparation of contents in a dispersing agent” is added to the precompressed coating, and coating for the top half of the tablet is added on top of the core material before a final compression step.

Tablets are frequently preferred and widely used compared to other dosage forms, because of their ease of administration, lower cost of manufacture and elegance. However, there is always a need for new and improved means for making compressed tablets more efficiently than is possible with existing technology. There is also a need for making compressed tablets of materials that do not readily form coherent bodies when compressed using known methods. For example, a need exists for methods and devices for forming compressed coated tablets of granulations or blends of core or coating material that do not readily form a coherent tablet using known methods. Finally, a need exists for methods and devices for forming compression coated tablets with structural and functional features not available in known compressed tablets. The present invention meets all of these needs, as demonstrated below.

### SUMMARY OF THE INVENTION

The present invention is directed to a tablet press for compression coating, where tablet cores as well as complete compression coated tablets, containing the cores, are manufactured during one turn of a single turret of a tablet press.

The tablet press of the present invention uses a novel upper punch comprising an outer hollow punch with a shaft extending from an outer head to an outer tip of the outer hollow punch, and a moveable core rod contained within the shaft. The outer tip of the outer hollow punch is designed to fit into an opening in the die. The end of the core rod closest to the outer tip is referred to as the rod tip, while the end of the core rod closest to the outer head is referred to as the rod head. The upper punch of the present invention is designed to shift between an extended configuration, where the rod tip is extended away from the outer tip of the outer hollow punch, and a retracted configuration, where the rod tip is substantially aligned with the outer tip, by movement of the core rod within the shaft. This feature of the upper punch makes it possible to use the same punch, in the extended and retracted configuration, to practice the tablet compression process of the present invention.

The present invention also relates to a tablet press comprising the upper punch, wherein the tablet press is designed to use the upper punch in both the extended and retracted configurations.

The present invention also relates to a process for compaction of particles into a tablet, wherein a core material is placed into a cup of coating material in a die; the core material is pressed into the cup with an upper punch in an extended configuration, and the cup is then compacted using an upper punch in a retracted configuration. The process of the present invention can be used to make a wide variety of different tablets, including coated tablets or partially coated tablets containing any one of a number of different biologically active agents. In one embodiment, the process is used to produce a tablet coated on all sides. In another embodiment, the process is used to produce tablets with layers of different colored coating and core material. In yet another embodiment, the process is used to produce tablets coated everywhere except in one or more spots on a surface where a different colored core material is exposed, giving the tablets a distinct appearance. In another embodiment, the process is used to produce tablets coated everywhere except on a top surface with a non-wettable or water resistant coating material and coated on the top surface with a different coating material, such as a bioadhesive agent or semipermeable membrane.

The present invention also relates to coated tablet, produced according to the process of the invention.

The present invention provides a process and materials for producing tablets of various core materials, including core materials that it would not otherwise be possible to form into tablets, using known methods or devices. Additional advantages of use of the process, products, and devices of the present invention will become apparent from the remaining disclosure, herein below.

### BRIEF DESCRIPTION OF THE DRAWINGS

FIG. 1 illustrates a side view of the upper punch, in an extended configuration in FIG. 1(A), and in a retracted configuration in FIG. 1(B). The head end of the upper punch is not shown.

FIG. 2 is a schematic illustrating use of an upper punch of the present invention to produce a coated compressed tablet.

FIG. 3 illustrates use of two different rollers to position the core rod within the shaft of a hollow upper punch, such that the upper punch is in an extended configuration when a solid roller is used, as shown in FIG. 3(A) (side view), and in a retracted configuration when a roller with a recessed groove is used, as shown in FIG. 3(B) (side view) and in FIG. 3(C) (front view).

FIG. 4 is a bar graph of crushing strength vs. compression force illustrating the crushing strength of tablets of the present invention containing poorly compactable core materials.

FIG. 5 is a graph illustrating release of theophylline from tablets of the present invention of various radial coating wall thickness. Coating=550 mg blend A, core=100 mg theophylline, lid=100, 150 and 200 mg blend A for thin (3.2 mm), intermediate (4.1 mm), and thick (4.8 mm) wall.

FIG. 6 is a graph showing release profiles from tablets of the present invention containing a core composed of a 2:1 blend of theophylline and PEO of different molecular weights. Cup=750 mg blend A, core=150 mg drug:polymer 2:1 blend, lid=200 mg blend A.

FIG. 7 is a graph showing release profiles of theophylline from tablets of the present invention composed of coatings of different molecular weights of PEO. Coating=750 mg polymer blends A, B or C, core=100 mg theophylline, lid=200 mg polymer blends A, B or C.

FIG. 8 is a graph showing release of theophylline from tablets of the present invention containing different amounts of PEO in the coating. Cup=750 mg of coating blend A or D, core=100 mg theophylline and lid=200 mg of coating blend A or D.

FIG. 9 is a side view of a tablet of the present invention, in which the core material is uncoated and exposed on the top surface of the tablet.

FIGS. 10A and 10B are side and top views of different embodiments of the tablet of FIG. 10, one with a circular shaped exposed core material. The core material is preferably a different color than the surrounding coating material.

FIG. 11 is a cross-sectional side view of another embodiment of the tablet of the invention, one in which a core of a drug and an osmotically active ingredient is compressed in a cup of insoluble polymer or lipid, forming bottom and side walls around the core, with a semipermeable polymer plug over the entire top surface of the core.

### DETAILED DESCRIPTION OF THE INVENTION

The present application provides a means of producing tablet dosage forms in the form of compression-coated tablets, including providing a means for forming such tablets from granulations or blends that do not readily form a compact. The compression-coating granulation or blend can be pre-formulated to provide desired functionalities to the coating. The only requirement for producing the dosage form described herein is that the core material should possess the ability to flow into a die during production. The method described can be used to produce compression-coated tablet dosage forms with minimal formulation development work.

In the process of the present invention, core material is placed into a cup of coating material, pressed into the cup using an upper punch with an extended tip, followed by compacting the cup containing the core material with an upper punch with a retracted tip, forming a tablet. The cup can either be preformed, or formed as an additional step of the process, using the upper punch in an extended configuration. At least one additional layer of the same or different coating material from the coating material used to make the cup can also be added prior to the final compacting step.

As used herein, the term “die” refers to a tool used in the production of a compressed tablet, with walls defining a cavity extending from a top surface a bottom surface of the die. The cavity of a die is designed to hold material to be pressed or compressed between an upper punch and a lower punch, when the two punches are inserted into opposite ends of the cavity. An end of a lower punch is present in the cavity of the die in each step of the process of the present invention.

As used herein, the term “upper punch in an extended configuration” refers to an upper punch in a configuration in which it is designed to be inserted into a die through a cavity opening in the top surface, as described above, wherein the end of the punch that is inserted into the die comprises a rod that extends away from the remainder of the punch. The upper punch in an extended configuration is designed such that when it is inserted into an opening in a die containing a material, it presses the center part of the material farther into the opening in the die than it does material around the perimeter of the opening.

As used herein, the term “upper punch in a retracted configuration” refers to an upper punch in a configuration which can be used to compact substantially all material in a die when inserted therein. See the remaining disclosure herein for specific examples of embodiments of the upper punch in a retracted or extended configuration, suitable for use in the processes and devices of the present invention.

The process of the present invention is preferably practiced using at least one upper punch and at least one die of the present invention, preferably using enough upper punches and dies to fill all the stations in a rotary tablet punch press designed to present the upper punches in both an extended tip configuration and a retracted tip configuration.

The equipment used for producing tablets of the present invention is suitably based on simple modifications to traditional rotary tablet presses, such as a traditional two- or three-layer tablet press. In the process of the present invention, core material is added to a cup of coating material in a die prior to being pressed by an upper punch in an extended configuration, followed by being compressed by an upper punch in a retracted position. The cup can be preformed before being introduced into the die. Alternatively, the cup is formed in the die by adding coating material to the die and compressing it therein using an upper punch in an extended position. Embodiments of the processes of the present invention do not require the separate formation of the core tablet. Thus, core material and coating material can be formed into a coherent tablet without separate formation of a core prior to coating.

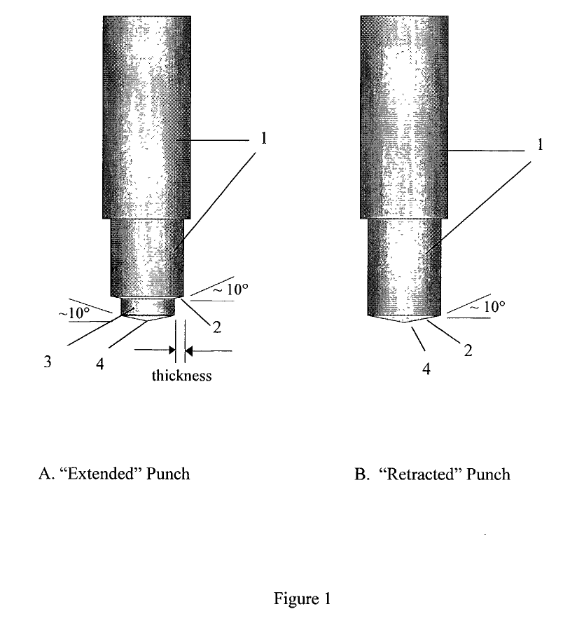

FIG. 1 illustrates the lower end of an embodiment of the upper punch of the present invention, in an extended configuration (FIG. 1A) and in a retracted upper punch configuration (FIG. 1B). FIG. 1A shows an outer hollow punch 1 with an outer tip 2 and a core rod 3 and rod tip 4, extending away from the outer tip 2 of outer hollow punch 1, forming an extended punch. The thickness of the surface of outer tip 5 determines the thickness of the walls of any cup formed using this embodiment of the upper punch. FIG. 1B shows outer hollow punch 1 in a retracted configuration, wherein outer tip 2 and outer edges of rod tip 4 are in alignment.

The rod tip and outer tip are preferably of a shape or of independent shapes that ensure formation of a coherent compressed tablet, when used in the process of the present invention. The most preferred shape of the outer tip and outer rod will vary depending upon the characteristics of the coating material, the core material, or the combination of coating and core material. A conical rod tip shape, where the cone angles away from the surface of the outer hollow punch, allows easy radial movement of materials being compressed at each stage of the process of the present invention, particularly when at least one material being compressed is in the form of a flowable powder. When conical in shape, the outer tip and rod tip can be at a different angles from one another, relative to the surface of the outer hollow punch.

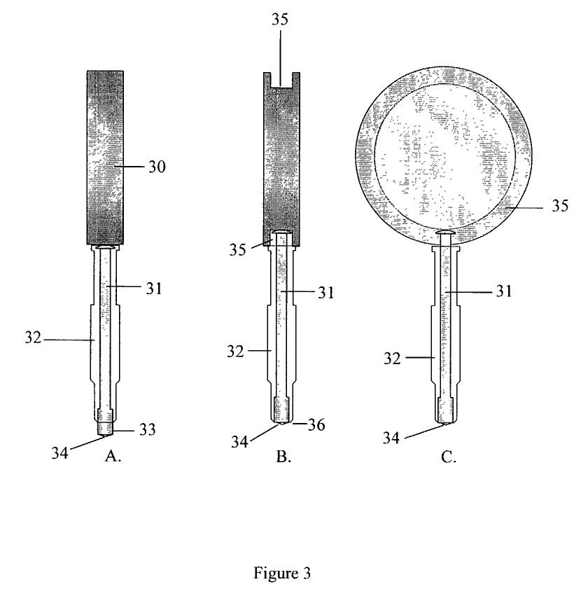

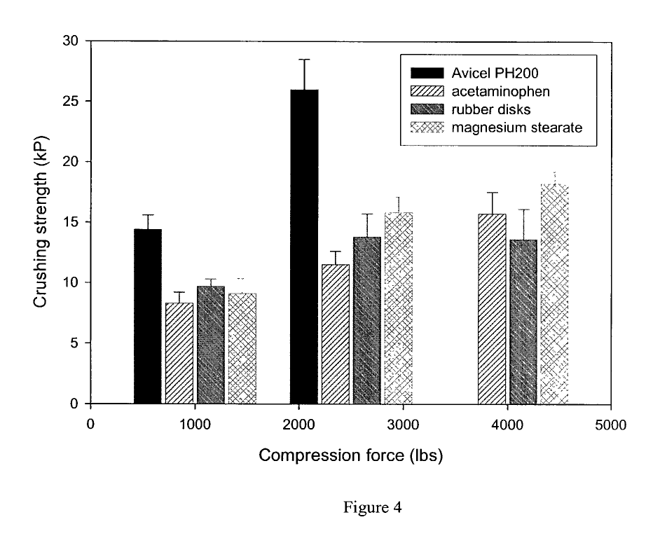

Both the outer tip and the rod tip are preferably conical in shape, with walls angling outward away from the outer hollow punch, at the same angle, producing a single conical punch tip shape when aligned with one another, when the upper punch is in a retracted configuration. An angled outer tip is also preferred to allow material deposited on the outer tip to slide (which may be assisted by machine vibration) into the cup after a tamping step has occurred (see, e.g., step 9 in FIG. 3, below). The optional tamping step also ensures that space is made for the core blend on the lip of the cup to slide into the cup. The angles of the outer tip and rod tip are preferably independently about 0° to about 45°, more preferably about 5° to about 25°, even more preferably about 5° to about 15°, even more preferably about 10°.

One such embodiment of an upper punch is illustrated in FIGS. 1A and B, where the surface of both the outer tip 2 and the surface of punch tip 4 are each shown as being conical in shape, angled away from the tip end of the outer hollow punch at a uniform angle of about 10°. When this embodiment of the upper punch is in a retracted configuration, the retracted tip formed when the outer tip 2 and punch tip 4 are aligned is also conical in shape and angled at about 10° away from the tip end of outer hollow punch 1.

We have found that when certain coating materials are pressed with an upper punch in an extended position in an attempt to form a cup, where the punch tip and outer tip are both flat, the coating blend does not tend to migrate in the radial direction to form a well-compacted wall. Such upper punches have been found to produce cups of such material with bottoms that are well compacted, while the walls of each cup were poorly formed. The resulting cups tended to crumble during withdrawal of the punch tip from the cup.

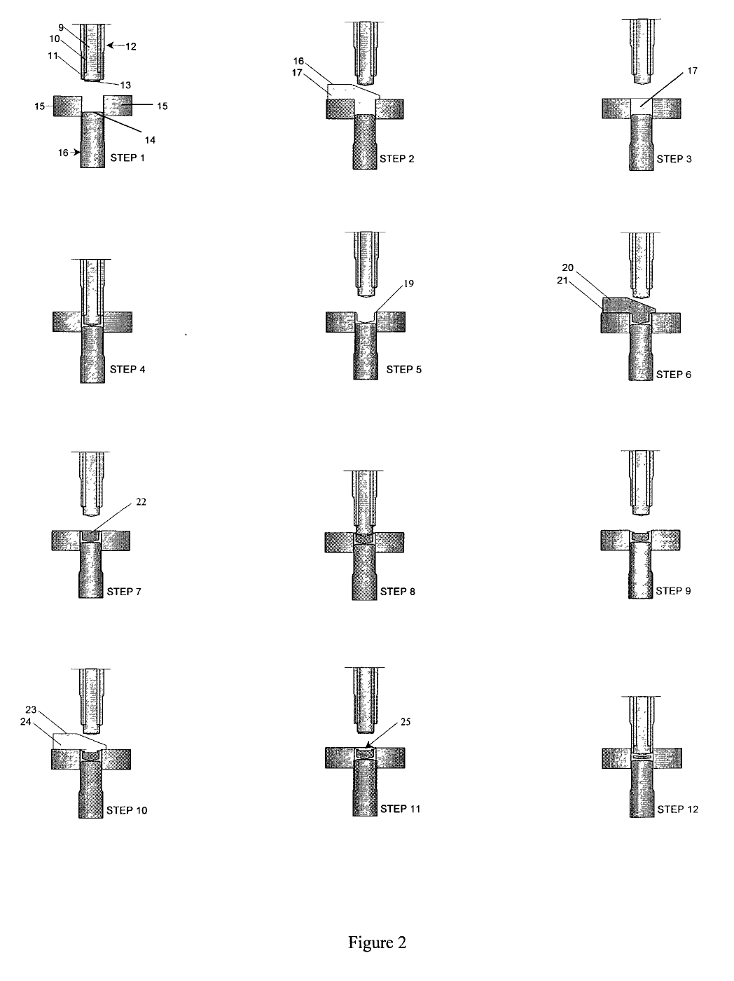

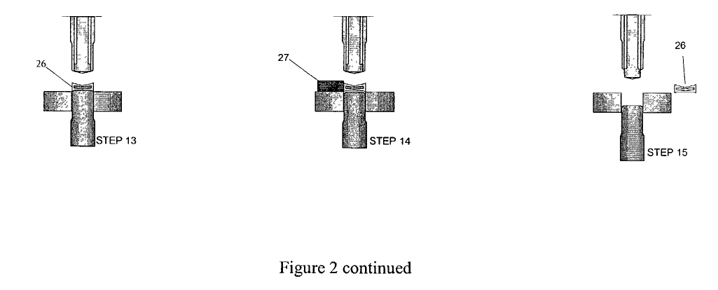

Use of a process of the invention to make a coated tablet on a three-layer tablet press is illustrated in FIG. 2, Steps 1-15. The same apparatus is used in all the steps. The apparatus is illustrated in detail in Step 1, that shows an upper punch 12 with a core rod 9, hollow shaft 10 formed by the walls of an outer hollow punch 11, wherein the core rod 9 includes punch tip 13. A lower punch 16, with an upper surface 14 is designed to be in contact with a surface of the cup of core material throughout the process. A die 15 is designed to receive one end of each of the upper and lower punches in order that materials can be compressed, at various steps of the process.

The remaining steps of FIG. 2 illustrate one preferred use of the device shown in Step 1 to make coated tablets according to a process of the present invention. In Step 2, first fill shoe 16 is used to introduce coating material 17 to the opening in the die. Step 3 shows the apparatus after the first fill shoe 16 has been removed, leaving the opening in the die filled with uncompressed coating material 17. Step 4 shows the upper punch in an extended configuration (i.e., with core rod 9 lowered relative to the outer hollow punch 11), being used to compress the core material 17 to form a cup 19 (shown in next step). Step 5 shows the apparatus after the upper punch has been raised, and removed from the die, with the cup 19 of core material in the die. In Step 6, core material 20 is introduced to the cup 19 through a second fill shoe 21. Step 7 shows the apparatus after the second fill shoe 21 has been removed, leaving uncompressed core material 22 in the cup. In Step 8, the upper punch, in the extended position, is inserted into the die to tamp the core material within the cup. In Step 9, the upper punch is removed from the die and excess material from the lip of the cup may slide on top of the tamped core material in cup 19. In Step 10, a third fill shoe 23 is used to introduce coating material 24 into the die, at least partially coating the surface of the core material 22. Step 11 shows the apparatus after the third fill shoe 23 has been removed, leaving an uncompressed coated tablet 25 in the die. In Step 12, the upper punch is shown in a retracted configuration (i.e., with core rod 9 raised to become level or substantially level with the end of the outer hollow punch closest to the lower punch.) Step 12 shows the apparatus with the core and coating material being compressed between the upper punch in retracted configuration and the lower punch, inside the channel of the die. Step 13, shows the apparatus, with the upper punch removed from the die, and the resulting compressed coated tablet 26 lifted up out of the die on the upper surface 14 of lower punch 16, after it has been moved upward through the opening in the die. In Step 14, a knock off plate 27 is shown positioned next to the compressed coated tablet 26, in preparation for removing the tablet from the apparatus. Step 15 shows the compressed coated tablet as it is being ejected from the apparatus.

FIG. 3 illustrates a means for alternating between the extended and retracted configurations of an upper punch with a hollow outer punch 32, and a core rod 33 with a tip 34 at one end and a head 35 at the other end that is moveable within the hollow outer punch 32, using contact of the head 35 with regular and recessed compression rollers. FIG. 3A is a side view of a traditional solid roller 30 in contact with the head 35 of the core rod 33 of one such upper punch 31, resulting in a configuration wherein the tip 34 is in an extended position. FIGS. 3B and 3C are side and front views, respectively of a recessed roller 37, and an upper punch 31 in a retracted position, due to the fact that the head 35 of the core rod 33 has moved from the surface of the roller into a recess contained therein, causing the tip 34 to be retracted into the hollow outer punch 32.

The embodiment of the process of the present invention described above provides a means of compression coating by simple modifications to a three-layer press. There are many advantages of the process of the present invention over traditional compression coating. Separate formation of a core is not required and therefore no transfer mechanism is required for the core. Similarly, centering of such a core on a bed of coating material is not a factor in this process. The elimination of this factor leads to better reproducibility of release profiles in controlled release applications. Also, there is no requirement for the core to be compactable, nor is adhesion between core and coating a prerequisite. A simple and non-compactable, but flowable granulation can be made into a tablet using the process of the present invention, as long as the coating blend is sufficiently compactable.

Suitable coating materials for use in the process of making compressed tablets of the present invention include, but are not limited to: polymers, such as polyethylene oxide, ethylcellulose and polymers and copolymers of acrylic acid, methacrylic acid and esters thereof; gelling polymers, such as hydroxypropyl methyl cellulose; lipids, such as glycerides of fatty acids, fatty acids, and fatty alcohols;; carbohydrates, such as lactose, sucrose, maltose, mannitol, sorbitol, starch, microcrystalline cellulose, and powdered cellulose; and dicalcium phosphate.

Other excipients can include, by way of illustration and not limitation, diluents, disintegrants, binding agents, adhesives, wetting agents, polymers, lubricants, glidants, substances added to mask or counteract a disagreeable taste or odor, flavors, dies, fragrances, and substances added to improve appearance of the composition.

Any bioactive substance can be incorporated in the invention. Preferably the bioactive agent is a drug, orally deliverable to mammals for example humans. The bioactive substance is preferably present in the core. However, it can also be present in at least one coating material used in the process of the invention.

The specific core and coating materials selected for use in the process of the present invention or in the tablets of the present invention will depend upon the particular characteristics desired in the end product. Where a characteristic appearance is desired to distinguish a tablet of the present invention from other such products, in order to prevent confusion by end users, one can use coating material and, at least one layer of the core material of a different color to produce the product. For example, a preformed cup of coating material of a different color from the core material is used, wherein the cup is designed to leave a spot of uncoated core material on the surface of the tablet after compression. The spot of uncoated core material can be any one of a number of symbols that distinguish the tablet of the present invention from tablets produced by other means. The spot of uncoated core material is preferably in the shape of a simple polygon, more preferably a triangle, a diamond, a square, or a circle.

Another way to produce a tablet of the present invention with a distinct appearance is to use at least one layer of coating that is differently colored from the cup coating material to make the tablet according to the process of the invention. When more than one additional layer is used, an additional layer of core material is preferably placed in between each layer of coating material.

It is also suitable to use cup coating material that is impenetrable to water or insoluble in water, such as a waxy material or an insoluble or hydrophobic polymer, to produce a tablet of the present invention that has distinct release properties. When such cup coating material is used with a core comprising an active agent and a gelling polymer, release of the active agent after ingestion of the tablet can be more precisely controlled. Specifically, the principal, if not the only surface of the tablet from which the active agent will be released will be from the surface of the gelling polymer. The gelling polymer matrix and release rate can be controlled, for example, by controlling the type and concentration of gelling polymer in the matrix or by adding excipients to the matrix that control release rate.

The tablet of the present invention is also suitably produced by compressing a core comprising an active agent and a bioadhesive polymer in such an insoluble or impenetrable cup, such as the one described above. This embodiment of the tablet of the present invention is suitably applied buccally with the bioadhesive core facing a buccal or sublingual surface. The cup coating material limits leaching of the active agent into the mouth while the active agent is being absorbed, thus, minimizing possible problems due to a bad tasting active agent.

The tablet of the present invention is also suitably produced by a process comprising steps of placing a layer of inner core material comprising a biologically active agent, such as a drug, and an osmotically active agent, in a cup comprising a coating material that is not water penetrable or water soluble, pressing the core material into the cup using an upper punch with an extended tip, adding a layer of an outer coating material that becomes semipermeable after formation of the compressed tablet, and compacting the cup using an upper punch with a retracted tip. The process for making the tablet of the present invention preferably further comprises an additional step of adding at least one layer of inner core material and outer core material, pressing each layer into the cup before the final compacting step. This embodiment of the tablet of the present invention provides drug release at a single point in time after ingestion. The process for making this product is more efficient and allows more design freedom than timed release gelatin capsules. Provided all other excipients are selected carefully, unlike gelatin capsules, a tablet of the present invention should be acceptable to strict vegetarians.

In addition to a tablet and a process for making a tablet, the present invention is also directed to a punch for compaction of particles into a tablet, comprising an outer hollow punch with a punch head at one end and a punch tip at the other end, walls of the outer hollow punch defining a hollow shaft extending from the punch head to the punch tip, and a moveable core rod contained within the hollow shaft, the punch comprising a rod tip at the end closest to the punch tip, and a rod head at the end closest to the punch head. The surface of the punch tip and the surface of the rod tip are preferably angled such as to form a conical surface, when the rod tip and punch tip are in alignment.

The present invention is also directed to a rotary tablet press comprising at least one upper punch of the present invention. The tablet press of the present invention preferably further comprises an upper punch of the present invention, a lower punch, and a die in each compression station of the punch. The upper punch of the present invention punch accords the preferred tablet press of the present invention considerable versatility and ease of manufacture compared to many other known means of manufacture of tablets.

The tablet press of the present invention preferably further comprises at least one roller with a circumferential groove designed to guide the movements of the core rod with respect to the outer hollow punch. More preferably, when the rod head of the upper punch is in the circumferential groove and the punch head is in contact with the surface of the roller outside the groove, the punch is in a retracted configuration. The tablet press preferably further comprises at least one roller without a circumferential groove. Preferably, when the rod head and upper punch are in contact with the roller without a circumferential groove, the rod tip extends beyond the surface of the punch tip, and the punch tip is in an extended configuration.

A tablet press suitable for use in making the tablets of the present invention can be made by making relatively simple modifications to a three-layer press by using a special hollow upper punch tooling somewhat similar to that described in Kilian, F., “New and Improved Method and Apparatus for the Production of Coated Tablets,” British Patent 464, p. 903 (1937), incorporated by reference herein. It is contemplated that such a tablet press will be very adaptable for use in the commercial production of tablets of the present invention. One example of commercial use of the modified upper punch to produce compression coated tablets of the present invention is illustrated in FIG. 2, discussed herein above.

Other advantages of the process, devices, and products of the present invention can also be envisaged such as the physical separation of two incompatible drugs within the same dosage form. This is commonly achieved by using multi-layer tablets or compression-coated dosage forms. Multi-layer tablets produced according to known methods, however, do not have the versatility of compression-coated tablets produced according to the method of the present invention. The present tablets can be easily modified to tailor release profiles of the drug from the dosage form. The process allows rapid development of customizable delayed or sustained-release dosage forms as illustrated herein below. The design of a specially adapted three-layer tablet press of the present invention, with modified tooling and compression rollers for the manufacture of tablets of the present invention at commercial scale is also presented herein.

Many other applications of the products, devices, and processes of the present invention can also be imagined. If a neat drug has good flow characteristics, it is preferably filled directly in the cup of core. The process of the present invention can also be modified to incorporate drug blends or multiple layers of the same or different drugs in the core or coating of compressed tablets. The present process thus provides an inexpensive and versatile alternative to filling granulations into pre-formed, conventional hard gelatin capsules; the drug or drug-containing particles are encapsulated within a previously formulated and prepared coating blend, instead of within a hard gelatin shell. As demonstrated herein, even the most poorly compactable substances can be presented as a tablet if a sufficiently compactable coating granulation is employed.

Various other applications of this technology are envisaged. It has already been shown that the release of the drug from the core can be readily modulated to produce timed-release dosage forms. Free drug crystals, drug-excipient blends, granules, microspheres or beads can be readily encapsulated inside the core of tablets of the present invention. Similarly, if a suitable polymer is used in the outer coating, it is possible to manufacture enteric, compression-coated dosage forms, as described in Fukui, E. et al., “Preparation of enteric coated timed-release press-coated tablets and evaluation of their function by in vitro and in vivo tests for colon targeting,” Int. J. Pharm. 204: 7-15 (2000). The process of the present invention makes it feasible to taste-mask unpleasant drugs and blind them for clinical studies, as described in Hadfield, P. J. et al., “The Potential Use of Compression coating in the Blinding of Clinical Trial Supplies,” Drug. Dev. Ind. Pharm. 13: 1877-1190 (1987). Orally dissolving tablets could also be easily formulated by using a coating blend consisting of rapidly-dissolving sugars with a core containing the drug as suitably taste-masked particles.

Combination drug products could also be formulated by using a coating blend containing a drug different from that in the core, as described in Oth, M. et al., “Arthrotec Part I: Conception of a product combining Misoprotol 200 mcg and EC Diclofenac Sodium 50 mg,” J. Pharm. Belg. 48(2): 153 (1993). Repeat-action tablets could be produced by including drug in the coating as well as the core blends respectively to achieve pulsatile delivery.

Materials and methods used in the examples, below are purely illustrative. Other suitable excipients materials and methods of practicing the present invention or making the compression coated tablets of the present invention could also be used. For the production of tablets of the invention, any pharmaceutically acceptable excipient could be used.

### EXAMPLES

### Example 1

Materials and Methods Used

Microcrystalline cellulose (Avicel PH200) was obtained from FMC Corporation (Philadelphia, Pa.). Polyethyleneoxide (hereinafter “PEO”) of different molecular weights was obtained from Union Carbide (Danbury, Conn.). Magnesium Stearate was obtained from Mallinckrodt (Phillipsburg, N.J.); theophylline and acetaminophen were obtained from Sigma Chemical Company (St. Louis, Mo.).

9/16″ tooling from a single punch press was modified to produce upper punches simulating extended and retracted configurations of an upper punch of the present invention. Three upper punches were machined as shown in FIG. 1A to simulate the shape of the lower end of an upper punch of the present invention in “extended” configuration. These three upper punches were machined to different dimensions to produce cups with a thick wall (4.8 mm), intermediate wall (4.1 mm), or a thin wall (3.2 mm). Another upper punch was machined as shown in FIG. 1B to mimic the shape of the lower end of the upper punch in a “retracted” configuration. The lower punch was flat faced. Each upper punch produced as described above was cylindrical in shape, with a conical shaped tip at the “extended” or “retracted” end described above, and flat faced at the opposite end.

### Example 2

Manufacture of Tablets Coated on All Sides

Upper and lower punches produced as described in Example 1 were placed in a Carver Press (Wabash, Ind.) with a die configured to fit each pair of punches. A carefully weighed amount of powder blend (hereinafter called coating blend) was placed in the die and compressed in the press at a known force with the type of tooling shown in FIG. 1a to produce a cup-shaped tablet (cup). Compositions of the coating blends used in this Example are listed in Tables 1 and 2, below.

TABLE 1
Coated Tablets Containing Different Core Materials

| | | | Primary | Secondary |
|---|---|---|---|---|
| Core material | Coating material | Core material | compression | compression |
| 40 mg Magnesium | 400 mg Avicel | 100 mg Avicel | 1000 lb | 1000, 2500 |
| Stearate | PH200 | PH200 | | and 4000 lb |
| 150 mg | 400 mg Avicel | 100 mg Avicel | 1000 lb | 1000, 2500 |
| Acetaminophen | PH200 | PH200 | | and 4000 lb |
| Rubber discs (1.8 | 400 mg Avicel | 100 mg Avicel | 1000 lb | 1000, 2500 |
| mm thickness and | PH200 | PH200 | | and 4000 lb |
| 10.2 mm diameter) | | | | |

TABLE 2
Coating Blends Used

| | | PEO | | | |
|---|---|---|---|---|---|
| Coating | PEO | amount | Fast-Flo | Magnesium | |
| Blends | Grades | (%) | Lactose 316 (%) | stearate (%) | Total |
| A | WSRN-1105 | 35.0 | 64.5 | 0.5 | 100.0 |
| B | WSRN-60K | 35.0 | 64.5 | 0.5 | 100.0 |
| C | WSRN-301 | 35.0 | 64.5 | 0.5 | 100.0 |
| D | WSRN-1105 | 50.0 | 49.5 | 0.5 | 100.0 |

The cup was left within the die and a known amount of either a model drug or blends containing drug was placed inside the cup and tamped lightly with the “extended” punch. A weighed amount of the coating blend was placed on top of the die contents, which was then compressed for a second time with the “retracted” punch at a known force to produce the final coated tablet.

### Example 3

Testing and Characterization of Coated Compressed Tablets

Drug release from tablets produced as described in Example 2 was studied in a USP dissolution tester type 2 (Vankel Industries, Edison, N.J.). Distilled water at 37° C. was used as the dissolution medium at a paddle speed of 100 rpm. The amount of drug released was monitored by automatic sampling of the dissolution media at regular intervals followed by UV spectrophotometric detection at 287 nm. The hardness of the tablets was measured on a hardness tester (Key International, Englishtown, N.J.).

### Example 4

Manufacture of Tablets with Three-layer Press

A modified three-layer press with an upper punch of the present invention is used to make compressed tablets. The tablet press includes a standard roller and a roller with a circumferential groove. The upper punch comprises an outer hollow punch and a hollow shaft containing an inner core rod, one end of which provides a retractable rod tip. The core rod runs along the length of the shaft of the outer hollow punch, and is longer than the shaft. When the upper punch passes under a compression roller in the tablet press, and the upper end of the rod becomes flush with the head of the hollow punch, the upper punch is in an extended configuration (e.g., FIG. 3A). When the upper punch passes under a specially modified roller with a circumferential groove (e.g., FIG. 3B), the rod head retracts into the recessed groove, such that the tip of the core rod and the annular tip of the hollow punch are flush with each other, placing the upper punch in a retracted configuration.

The compressed tablets are manufactured on the modified three-layer tablet press, described above, according to the 15 step procedure illustrated in FIG. 2, above. The sequence illustrated in FIG. 2 consists of six distinct stages, three filling and three compression stages. Steps 1 through 5 depict the formation of the cup. At the first filling station, the coating granulation is fed into the die and in the first compression event the extended tip of the punch forms a cup, which remains within the die. The cam track on the tablet press (not depicted in FIG. 3) is adjusted such that the cup is formed very close to the top of the die, i.e., the lip of the cup should be close to the upper surface of the die. At the second filling station, the core blend (drug or granulation containing drug) to be encapsulated in the tablet is fed into the cup. At the second compression station, the core material is tamped lightly by the upper punch, which remains in the extended position as shown in steps 7 through 9. In step 10, at the third filling station, the coating granulation is again layered over the die contents to serve as a lid. At the final compression station the tip retracts because the upper punch passes under the special recessed/grooved roller; this allows the entire contents of the die to be compressed to form a coated tablet of the present invention, as shown in steps 10 through 15.

### Example 5

Production of Tablets from Poorly Compactable Materials

The simulated upper punches in “extended” and “retracted” configurations produced as described in Example 1 were used to produce compression coated tablets on a Carver press using a flat-faced lower punch and a corresponding die, as described in Example 2. The tablets were produced using 1000 lb compression force for the cup, followed by manual tamping of the core material, and final compression of the die contents (cup, core and lid) at various compression forces. A cup and compression coated tablet of the present invention was made from Avicel PH200 using tooling from FIG. 1A.

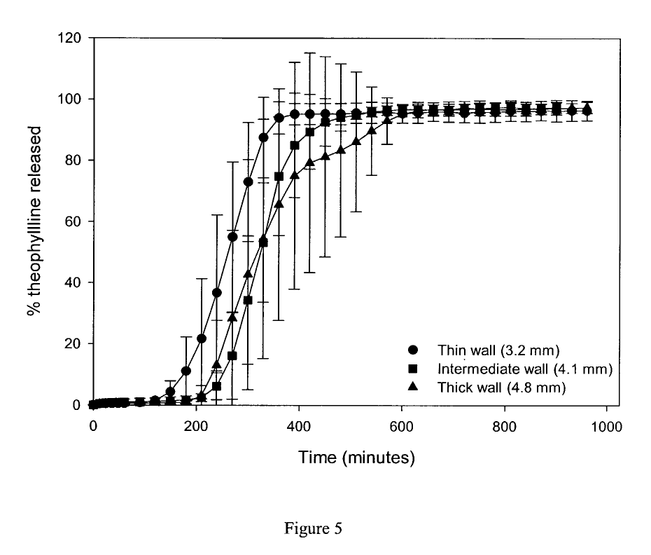

Using the above tooling, compression coated tablets of the present invention containing three poorly compactable substances, magnesium stearate, acetaminophen and rubber discs were prepared on a Carver press. In each instance, a highly compactable substance, Avicel PH200 was used as the coating blend. The cups were formed at a force of 1000 lb and the final compression was done at either 1000, or 2500, or 4000 lb. The amounts of coating, lid and core material for each of these experiments is shown in table 1. FIG. 5 shows the crushing strength of these compacts at various compression forces compared to compacts made from pure Avicel PH200. Compacts made from Avicel were also made in three-step process similar to the manufacturing process described in Example 2, above.

The process used in this case demonstrated the ability to form intact compacts from poorly compactable materials. The crushing strength of acetaminophen and magnesium stearate compacts increased with an increase in compression force from 1000 to 4000 lb, but was significantly lower than that of compacts made from pure Avicel PH200 at the same compression force. Avicel compacts made at 4000 lb had hardness values beyond the range of the hardness tester. The crushing strength of rubber compacts did not appear to increase with an increase in compression force beyond 2500 lb. This is suggestive of higher elastic energy stored within the tablet at higher compression forces, which reduces the strength of the compact.

### Example 6

Production of Controlled Release Tablets

Compression coated tablets of the present invention were prepared on a Carver press, using coating blends containing different amounts and types of PEO as a rate-controlling hydrophilic polymer, as described in Kim, C. J., “Drug release from compressed hydrophilic PEO-WSR tablets,” J. Pharm. Sci. 84: 303-306 (1995). Fast-Flo Lactose 316 was used as a filler and 0.5% magnesium stearate was added to each blend to serve as a lubricant. The core material was composed of either pure theophylline or a blend of theophylline with PEO. The various coating blends that were used to prepare compressed tablets of the present invention are shown in table 2, above.

The effect of the thickness of the wall of the cup on the release of theophylline from the resulting tablets is shown in FIG. 5. Three different wall thicknesses of 3.2 mm, 4.1 mm and 4.8 mm could be produced by using upper punches machined to different dimensions as described in the materials and methods section. Increasing amounts of coating blend for the cup and the lid were used for punches with increasing wall thickness. Tablets with a thin wall began to release drug earlier (˜120 min) than tablets with an intermediate and thick wall (˜200 min).

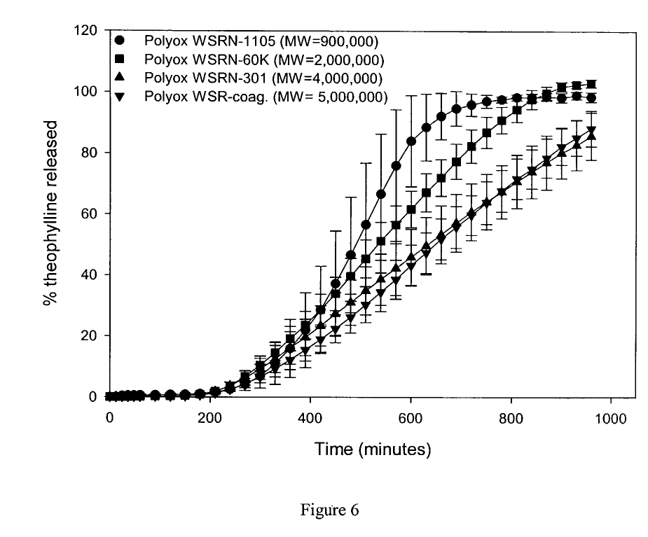

In an attempt to obtain better control of the delay (lag) period before release of the drug begins, the molecular weights of the polymer in the coating were varied. FIG. 6 shows that coatings containing PEO of MW 2,000,000 took almost twice as long for the dissolution medium to breach the coating and for drug release to begin. In the case of PEO with MW 4,000,000, it appears that the coating was not breached for the release period studied; instead, the drug seems to be diffusing through the coating barrier.

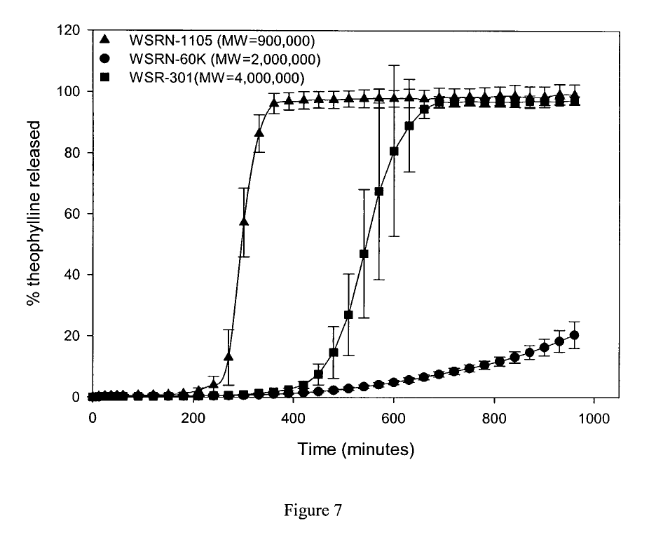

Another approach to control the delay period before drug release begins was to use different concentrations of polymer in the coating blend. FIG. 7 compares coatings containing 35% and 50% of PEO of MW 900,000. As expected, higher concentrations of the polymer were able to delay the release of the drug for a longer period of time.

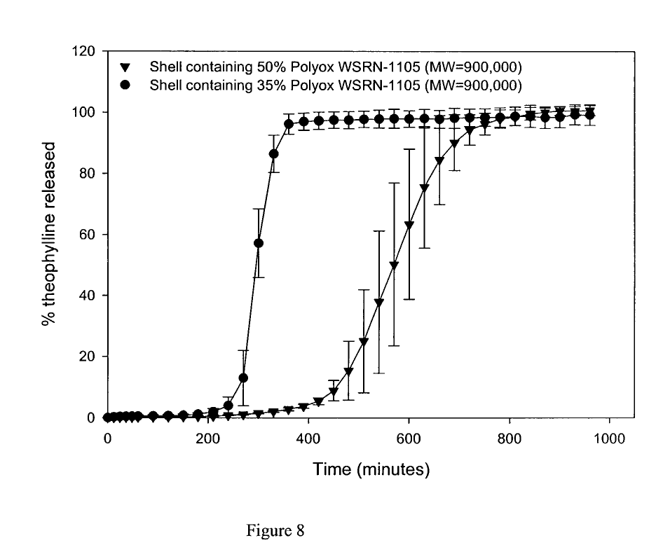

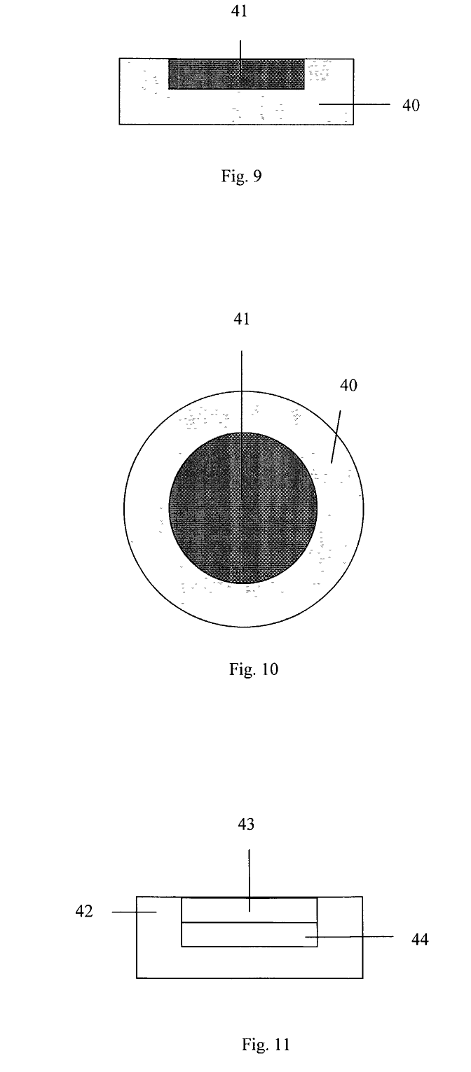

The effect of including polymer in the core blend in addition to incorporation in the coating blend was studied as shown in FIG. 8. The coating blends in these experiments were composed of PEO of MW 900,000 while the cores contained 2:1 blends of drug and PEO of different molecular weights. In each case, the release of the drug began at approximately the same time, but was followed by slower release of drug at relatively constant rates. As expected, compressed tablets produced as described above containing polymers of lower MW in the core released drug slightly faster. There was however, no appreciable difference in the release rate of drug from tablets containing PEO of MW 4,000,000 and 5,000,000.

We claim:

1. A process for production of a tablet comprising the steps of:
a) placing core material into a cup in a die, wherein the cup is comprised of coating material, and
b) pressing the core material into the cup using an upper punch in an extended configuration, and
c) compacting the cup using an upper punch in a retracted configuration, thereby forming the tablet.

2. The process of claim 1, wherein the coating material and core material are a different color.

3. The process of claim 2, wherein at least a portion of the core material is exposed on a surface of the tablet.

4. The process of claim 1, wherein the coating material is impermeable to water and insoluble in water.

5. The process of claim 4, wherein the core comprises an active agent and a gelling polymer.

6. The process of claim 4, wherein the core comprises an active agent and a bioadhesive polymer.

7. The process of claim 4, wherein the core placed in the cup in step (a) is an inner core material comprising a drug and an osmotically active agent, the process further comprising layering a semipermeable outer core material on the inner core material after step (b), and repeating step (b) prior to step (c).

8. The process of claim 1 further comprising adding at least one additional layer of coating material to the cup prior to compacting the cup in step (c).

9. The process of claim 1, further comprising forming the cup by compacting the coating material with the upper punch in an extended configuration in the die prior to step (a).

10. The process of claim 1 wherein the upper punch comprises an outer hollow punch with walls defining a hollow shaft and a moveable core rod contained within the hollow shaft, wherein movement of the core rod within the shaft alternately results in the upper punch being in an extended configuration or in a retracted configuration.

11. The process of claim 1, the core material comprising material selected from the group consisting of free drug crystals, drug excipient blends, granules, microspheres, and beads.

12. The process of claim 1, the coating material comprising an excipient selected from the group consisting of a polymer selected from a hydrophilic polymer, polyethylene oxide, ethylcellulose, a polymer of acrylic acid, a copolymer of acrylic acid, methacrylic acid, esters of methacrylic acid, and a gelling polymer such as hydroxypropyl methyl cellulose; a lipid such as a glyceride of a fatty acid, a fatty acid, a fatty alcohol, an alkane; a carbohydrate such as, lactose, sucrose, maltose, mannitol sorbitol, starch, microcystalline cellulose and powdered cellulose; and dicalcium phosphate.

13. The process of claim 1 that is carried out in a tablet press.

14. An upper punch for production of a tablet comprising:
an outer hollow punch with an outer tip at one end and an outer head at another end, walls of the outer hollow punch defining a hollow shaft extending from the outer tip through the outer hollow punch to the outer head; and
a core rod contained within the hollow shaft, the rod having a rod tip at an end closest to the outer tip and a rod head at an end closest to the outer head, the core rod being moveable within the shaft.

15. The punch of claim 14, wherein the punch tip and the rod tip are angled as to form a conical surface, when the punch tip and the rod tip are aligned.

16. A tablet press comprising an upper punch comprising:
an outer hollow punch with an outer tip at one end and an outer head at another end, walls of the outer hollow punch defining a hollow shaft extending from the outer tip through the outer hollow punch to the outer head; and
a core rod contained within the hollow shaft, the rod having a rod tip at an end closest to the outer tip and a rod head at an end closest to the outer head, wherein movement of the core rod within the shaft alters the configuration of the upper punch from an extended configuration to a retracted configuration.

17. The tablet press of claim 16, wherein the tablet press is a rotary tablet machine.

18. The tablet press of claim 16, further comprising a means for directing movement of the core rod within the hollow shaft.

19. The tablet press of claim 18, wherein the means for directing movement of the core rod within the hollow punch comprises a roller with a circumferential groove, and the depth of said circumferential groove controls the position of the core cod with respect to the rest of the punch.

20. The tablet press of claim 19, further comprising at least one roller without a circumferential groove.

21. A tablet manufactured according to a process comprising the steps of:
a) placing core material into a cup comprised of coating in a die, wherein the cup is comprised of coating material, and
b) pressing the core material into the cup using an upper punch in an extended configuration with an extended tip, and
c) compacting the cup using an upper punch in an extended configuration with a retracted tip, thereby forming a tablet comprising a core and a coating.

22. The tablet of claim 21, wherein the coating material and core material are a different color.

23. The tablet of claim 22, wherein the cup is designed to produce a tablet with a surface wherein a portion of the core material is exposed.

24. The tablet of claim 21, wherein the coating material is not water penetrable or water soluble.

25. The tablet of claim 24, wherein the core comprises an active agent and a gelling polymer.

26. The tablet of claim 24, wherein the core comprises an active agent and a bioadhesive polymer.

27. The tablet of claim 24, wherein the core placed in the cup in step (a) is an inner core material comprising a drug and an osmotically active agent, the process further comprising layering a semipermeable outer core material on the inner core material after step (b), and repeating step (b) prior to step (c).

28. The tablet of claim 21, further comprising forming the cup by compacting the coating material with the upper punch with an extended tip in the die prior to step (a).

29. The tablet of claim 21, having a weight from about 0.01 g to about 100 g.

30. The tablet of claim 21, wherein the tablet disintegrates after immersion in water.

31. The tablet of claim 21, where the core comprises a poorly compactable substance.

32. The tablet of claim 21, wherein the tablet is in the form of a controlled release tablet.

33. The tablet of claim 21, wherein the tablet is enteric coated.

34. The tablet of claim 21, the core and the coating each comprising at least one bioactive substance.

35. The tablet of claim 34, wherein the tablet is designed for timed release of the at least one bioactive substance after ingestion.

36. The tablet of claim 34, wherein the tablet exhibits pulsatile release of the at least one bioactive substance after ingestion.

37. The tablet of claim 34, wherein the core comprises a first portion of the at least one bioactive substance and the coating comprises a second portion of the least one bioactive material in the coating, the first portion and the second portion having different release rates after ingestion.

38. The tablet of claim 21, where the core material comprises material selected from the group consisting of free drug crystals, drug excipient blends, granules, microspheres, and beads.

39. The tablet of claim 21, comprising at lease one bioactive substance with unpleasant taste or smell in the core.

40. The tablet of claim 39, the coating comprising at least one rapidly dissolving substance.

41. The tablet of claim 40 wherein the rapidly dissolving substance comprises at least one sugar.

42. A process for production of a tablet comprising the steps of:
a) forming a cup of core material by pressing the core material in a die using an upper punch in an extended configuration;
b) placing core material into the cup in the die, and
c) pressing the core material into the cup using an upper punch in an extended configuration, and
d) compacting the cup and core material using an upper punch in a retracted configuration, thereby forming the tablet.

43. The process of claim 42, wherein the upper punch comprises an outer hollow punch with walls defining a hollow shaft and a moveable core rod contained within the hollow shaft, wherein movement of the core rod within the shaft alternately generates the upper punch in the extended configuration and the upper punch in the retracted configuration.

44. The process of claim 42, wherein the core material is in a flowable particulate form.

45. The process of claim 42, the core material comprises material selected from the group consisting of free drug crystals, drug excipient blends, granules, microspheres, and beads.

46. The process of claim 42, the coating material comprising an excipient selected from the group consisting of a polymer selected from a hydrophilic polymer, polyethylene oxide, ethylcellulose, a polymer of acrylic acid, a copolymer of acrylic acid, methacrylic acid, esters of methacrylic acid, and a gelling polymer such as hydroxypropyl methyl cellulose; a lipid such as a glyceride of a fatty acid, a fatty acid, a fatty alcohol, an alkane; a carbohydrate such as, lactose, sucrose, maltose, mannitol sorbitol, starch, microcystalline cellulose and powdered cellulose; and dicalcium phosphate.

47. The process of claim 42 that is carried out in a tablet press.
